(19) United States
(12) Patent Application Publication
(10) Pub. No.: US 2014/0236010 A1
(43) Pub. Date: Aug. 21, 2014

(54) ULTRASONIC DIAGNOSTIC APPARATUS AND MEDICAL IMAGE PROCESSING APPARATUS

(71) Applicants: Kabushiki Kaisha Toshiba, Minato-ku (JP); Toshiba Medical Systems Corporation, Otawara-shi (JP)

(72) Inventor: Kenshi NAKANO, Otawara-shi (JP)

(73) Assignees: Kabushiki Kaisha Toshiba, Minato-ku (JP); Toshiba Medical Systems Corporation, Otawara-shi (JP)

(21) Appl. No.: 14/266,017

(22) Filed: Apr. 30, 2014

Related U.S. Application Data
(63) Continuation of application No. PCT/JP2013/074596, filed on Sep. 11, 2013.

(30) Foreign Application Priority Data
Sep. 25, 2012 (JP) 2012-211539

(51) Int. Cl. A61B 8/08; A61B 8/00

(52) U.S. Cl. CPC A61B 8/5223; A61B 8/463
USPC 600/440

(57) ABSTRACT

An ultrasonic diagnostic apparatus according to an embodiment includes an image acquisition unit, a measurement unit, an estimation unit, and a display unit. The image acquisition unit acquires an ultrasonic image representing information inside an object. The measurement unit executes measurement on the ultrasonic image. The estimation unit estimates a measurement region associated with the measurement based on the ultrasonic image. The display unit displays the estimated measurement region together with the ultrasonic image.

### CROSS-REFERENCE TO RELATED APPLICATIONS

This application is a Continuation application of PCT Application No. PCT/JP2013/074596, filed Sep. 11, 2013 and based upon and claims the benefit of priority from the Japanese Patent Application No. 2012-211539, filed Sep. 25, 2012, the entire contents of all of which are incorporated herein by reference.

### FIELD

Embodiments described herein relate generally to an ultrasonic diagnostic apparatus and medical image processing apparatus which process the ultrasonic image generated based on the data acquired by transmitting and receiving ultrasonic waves to and from an object.

### BACKGROUND

The measurement executed by an ultrasonic diagnostic apparatus can be broadly classified into fundamental measurement and application measurement. Fundamental measurement is the operation of simply measuring a distance, area, time, velocity, acceleration, and the like without providing in advance any clinical meaning on the ultrasonic diagnostic apparatus. In contrast, application measurement is measurement to be executed upon assigning (explicitly indicating) a clinical meaning on the ultrasonic diagnostic apparatus at the time of the execution of measurement.

That is, when executing application measurement, the user decides a measurement region, measurement items, and the like (LVPWT: Left Ventricle Posterior Wall Thickness) or the like) in advance and then operates a predetermined operation button (the “LVPWT” button in this case) on the operation input unit of an ultrasonic diagnostic apparatus. In this case, a measurement result (measurement value) is assigned with the clinical meaning “left ventricle posterior wall thickness” (assigned with an identifier). This identifier can be recorded so as to be discriminated by apparatuses available from other makers.

More specifically, the data acquired by application measurement is generally recorded in a predetermined format. As this predetermined format, for example, there is available the “DICOM-SR standard” (DICOM=Digital Imaging and Communications in Medicine, SR=Structured Reporting).

The data output in a predetermined format such as the DICOM-SR standard allows apparatuses available from other makers to discriminate a measurement result of the data.

The DICOM-SR standard cannot set items without any clinical meanings. For this reason, it is not possible to output any data obtained by fundamental measurement according to the DICOM-SR standard. In addition, it is difficult to specify a position in an object displayed on an ultrasonic image from the display of the image.

Recently, in hospitals, the ultrasonic images acquired by ultrasonic image diagnostic apparatuses and the measurement results obtained on images are transmitted to a report server and archived in it according to the DICOM-SR standard, and are used for diagnosis, as needed. In addition, measurement results are often transmitted to the report server according to the DICOM-SR standard to create diagnostic reports on the report server.

However, the data obtained by fundamental measurement are merely data added with maker-specific identifiers indicating fundamental measurement values and units of measurement. Therefore, the data obtained by fundamental measurement do not allow their measurement values to be reflected in a diagnostic report until a clinical meaning is assigned to each measurement value. For this reason, when performing re-diagnosis by using such an image, the omission of memory by a person in charge of medical care or personnel change makes it impossible to perform proper re-diagnosis.

As described above, although fundamental measurement has a merit of easily performing measurement, an apparatus which has received measurement results cannot discriminate the clinical meanings of the measurement results. The clinical meanings of the measurements depend only on memory of medical personnel who has performed examinations.

Under the circumstances, there is proposed a technique of assigning clinical meanings (identifiers) to the data obtained by fundamental measurement.

However, there is no disclosure about any specific processing method of assigning clinical meanings to the data obtained by fundamental measurement. Under the above circumstances, demands have recently arisen for a technique of assigning clinical meanings to the data obtained by fundamental measurement by simple processing and outputting the data in a predetermined format (e.g., the DICOM-SR standard) as in the case of application measurement.

### BRIEF DESCRIPTION OF THE DRAWINGS

FIG. 1 is a block diagram showing the system configuration of an ultrasonic diagnostic apparatus according to the first embodiment.

FIG. 2 is a flowchart showing fundamental measurement processing executed by the ultrasonic diagnostic apparatus according to the first embodiment.

FIG. 3 is a view showing an example of a 4-chamber image at cardiac end-diastole according to the first embodiment.

FIG. 4 is a view showing a display example of an identifier assignment processing window according to the first embodiment.

FIG. 5 is a view showing an example of menu items displayed as pull-down menus of a measurement region setting window and measurement item setting window according to the first embodiment.

FIG. 6 is a flowchart of fundamental measurement processing executed by an ultrasonic diagnostic apparatus according to the second embodiment.

FIG. 7 is a view showing an example of measurement region estimation processing based on a pattern matching technique according to the second embodiment

### DETAILED DESCRIPTION

An ultrasonic diagnostic apparatus according to an embodiment includes an image acquisition unit, a measurement unit, an estimation unit, and a display unit.

The image acquisition unit acquires an ultrasonic image representing information inside an object.

The measurement unit executes measurement on the ultrasonic image.

The estimation unit estimates a measurement region associated with the measurement based on the ultrasonic image.

The display unit displays the estimated measurement region together with the ultrasonic image.

An ultrasonic diagnostic apparatus and medical image processing apparatus according to an embodiment of the present invention will be described below with reference to the accompanying drawings.

### First Embodiment

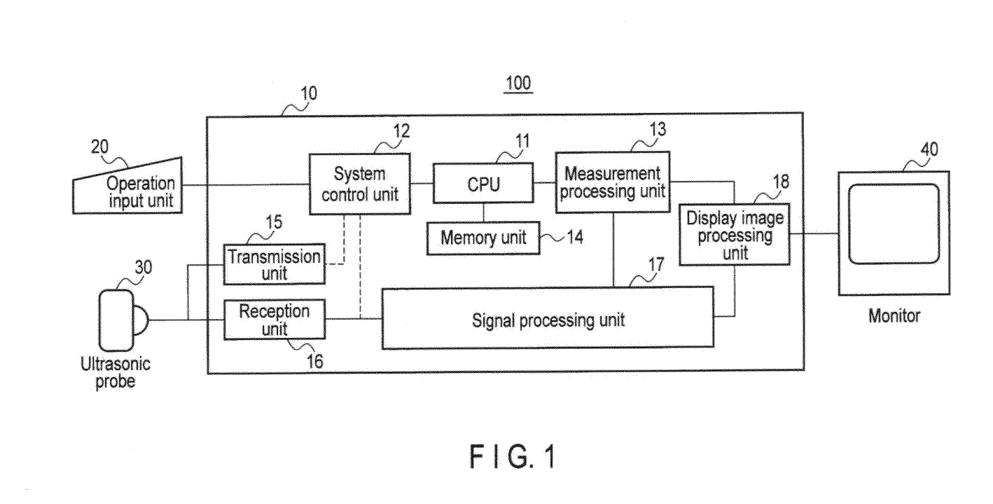

FIG. 1 shows the system configuration of an ultrasonic diagnostic apparatus according to the first embodiment. As shown in FIG. 1, an ultrasonic diagnostic apparatus 100 includes an ultrasonic diagnostic apparatus main body unit 10, an operation input unit 20, an ultrasonic probe 30 which applies ultrasonic waves to an observation region and receives echoes from the region, and a monitor 40 which displays the ultrasonic diagnostic image output from the ultrasonic diagnostic apparatus main body unit 10.

The ultrasonic diagnostic apparatus main body unit 10 includes a CPU 11, a system control unit 12, a measurement processing unit 13, a memory unit 14, a transmission unit 15, a reception unit 16, a signal processing unit 17, and a display image processing unit 18.

The CPU 11 comprehensively controls the overall ultrasonic diagnostic apparatus 100.

The CPU 11 controls the system control unit 12. The system control unit 12 mainly controls the hardware function of the ultrasonic diagnostic apparatus main body unit 10.

The measurement processing unit 13 performs various types of measurement from acquired ultrasonic diagnostic images. That is, the measurement processing unit 13 also functions as a fundamental measurement unit which executes fundamental measurement on an ultrasonic diagnostic image.

The memory unit 14 records/stores programs for control processing and measurement processing results.

The transmission unit 15 and the reception unit 16 transmit and receive ultrasonic signals controlled by the system control unit 12.

The signal processing unit 17 forms an ultrasonic diagnostic image by amplifying a reception signal and processing the signal in accordance with a scan scheme. That is, the signal processing unit 17 functions as an image acquisition unit which acquires an ultrasonic diagnostic image representing information inside an object, together with the transmission unit 15 and the reception unit 16.

The display image processing unit 18 combines the display image of the ultrasonic diagnostic image obtained by the signal processing unit 17 and the image measurement result obtained by the measurement processing unit 13 and outputs the result to the monitor 40.

The operation input unit 20 includes a pointing device such as a mouse or trackball, a keyboard, and a console panel including a TCS (Touch Command Switch).

The ultrasonic probe 30 is a device (probe) which transmits and receives ultrasonic signals applied and reflected to and from an object based on driving signals from the transmission unit 15 controlled by the system control unit 12. The ultrasonic probe 30 includes piezoelectric elements such as piezoelectric ceramic elements as electromechanical reversible conversion elements. The ultrasonic probe 30 converts a supplied pulse driving voltage into an ultrasonic pulse signal, transmits the signal in a desired direction in a scan region in an object, and converts an ultrasonic signal reflected from the object into a voltage echo signal corresponding to the signal.

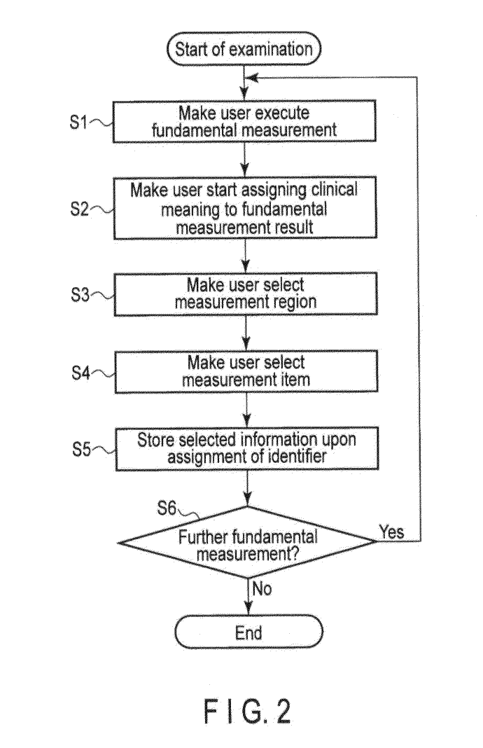

FIG. 2 is a flowchart of fundamental measurement processing executed by the ultrasonic diagnostic apparatus according to the first embodiment.

First of all, the user operates the operation input unit 20 while referring to the display on the monitor 40 to input the patient ID of an object to the ultrasonic diagnostic apparatus 100 and select a region called a body mark with respect to a region to be observed. The ultrasonic diagnostic apparatus 100 automatically sets an image mode suitable for a diagnostic purpose on the region, e.g., a B-image mode (tomographic image mode), an M-image mode (an image mode of displaying a moving distance), or a D-image mode (Doppler mode), image quality settings such as a luminance, contrast and a resolution, a measurement mode (to be described later), measurement items (to be described later), and the like.

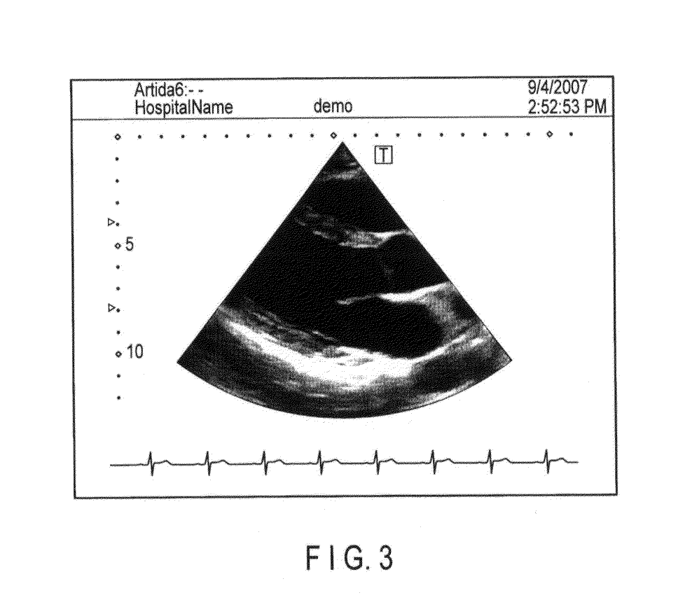

Subsequently, in order to obtain an ultrasonic diagnostic image, the user brings the ultrasonic probe 30 into contact with a measurement region of an object and starts acquiring ultrasonic images. The ultrasonic diagnostic apparatus 100 displays the ultrasonic diagnostic image on the monitor 40. Assume that in this case, the apparatus acquires a 4-chamber image (an image indicating a left ventricle posterior wall thickness, left ventricle inner diameter, interventricular septal thickness, and right ventricle inner diameter) at cardiac end-diastole like that shown in FIG. 3.

When an image sufficient for the execution of measurement on an observation region (the ultrasonic image shown in FIG. 3 in this case) is displayed on the monitor 40, the user executes “fundamental measurement” on the observation region (step S1).

The user starts fundamental measurement in step S1 by performing operation for the execution of the fundamental measurement using the keyboard, TCS, and mouse or pointing device such as a trackball constituting the operation input unit 20 of the ultrasonic diagnostic apparatus 100. The CPU 11 selects a measurement sub-program in accordance with this operation and operates.

In this case, the user uses the operation input unit 20 to perform the operation of displaying the “identifier assignment processing window W” for the assignment of a clinical meaning (the assignment of an identifier) to a fundamental measurement result (step S2). That is, if the user does not perform the operation in step S2, the apparatus performs general fundamental measurement.

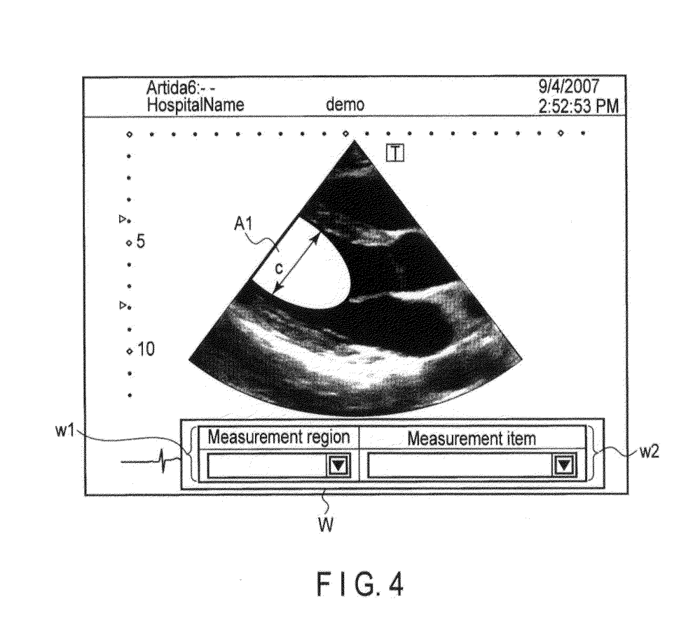

In accordance with the operation in step S2, the CPU 11 causes the monitor 40 to display the “identifier assignment processing window W” for the assignment of a clinical meaning (the assignment of an identifier) to a fundamental measurement result, together with the corresponding ultrasonic image (for example, superimposing the image) as shown in FIG. 4. In this case, as shown in FIG. 4, the apparatus measures a distance c in a region A1.

The identifier assignment processing window W includes a “measurement region setting portion w1” for setting a fundamental measurement execution target region (to be simply referred to as a “measurement region” hereinafter) and a “measurement item setting portion w2” for setting an item to be measured (to be referred to as a “measurement item” hereinafter).

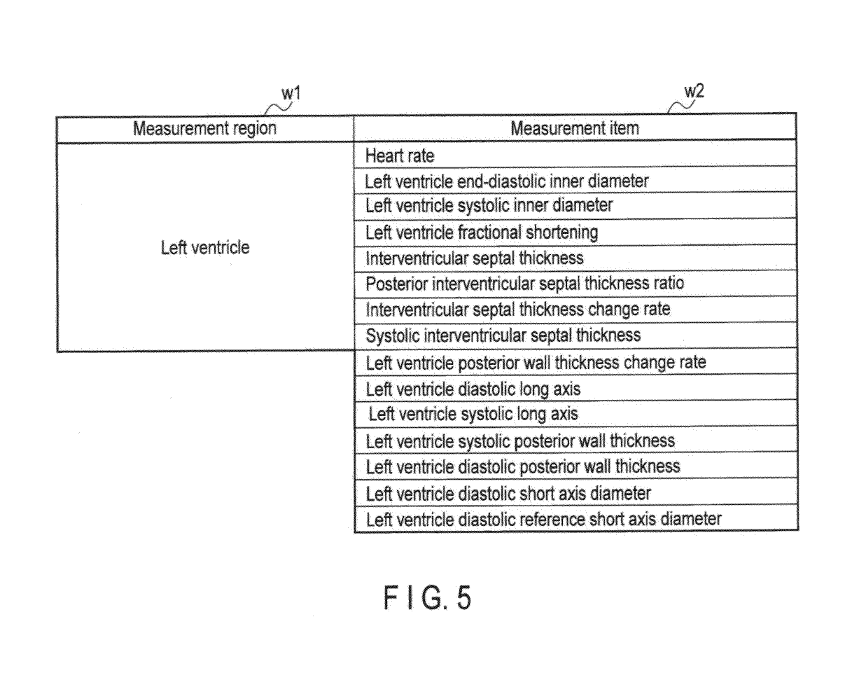

The measurement region setting portion w1 is an operation window in a pull-down menu form (to be described in detail later with reference to FIG. 5) in which measurement regions on which fundamental measurement can be executed are set as a plurality of menu items (measurement region candidates). The user selects a measurement region in this fundamental measurement from the menu items of the pull-down menu of the measurement region setting portion w1 by using the operation input unit 20 (step S3).

In general, in the ultrasonic diagnostic apparatus, the user roughly designates a region such as an “abdominal region” or “adult heart” as a measurement region at the start of examination (to be referred to as an “examination start initial setting” hereinafter) by using the operation input unit 20. If, therefore, the user sets, for example, the “circulatory system” as an examination start initial setting, the CPU 11 may present menu items of the measurement region setting portion w1 upon narrowing them down to those for measurement regions of the circulatory system in advance.

The measurement item setting portion w2 is an operation window in a pull-down menu form (to be described in detail later with reference to FIG. 5) in which measurement items which can be executed in fundamental measurement are set as a plurality of menu items (measurement item candidates). The user selects a measurement item in this fundamental measurement from the menu items of the pull-down menu of the measurement item setting portion w2 by using the operation input unit 20 (step S4).

In this case, the apparatus displays only measurement items corresponding to the measurement region selected by the measurement region setting portion w1 as menu items displayed as the pull-down menu of the measurement item setting portion w2. In other words, the CPU 11 displays only measurement items associated with the measurement region selected by the measurement region setting portion w1 as menu items of the pull-down menu of the measurement item setting portion w2.

FIG. 5 is a view showing an example of the menu items displayed as the pull-down menu of the measurement region setting portion w1 and measurement item setting portion w2. In this case, since the apparatus is to perform fundamental measurement on the distance c in the region A1 at cardiac end-diastole as shown in FIG. 4, the user selects the “left ventricle” corresponding to the region A1 as a measurement region, and selects the “left ventricle end-diastolic inner diameter” corresponding to the distance c from the menu items corresponding to the left ventricle as a measurement item.

The user then performs the operation of storing the measurement result obtained by this fundamental measurement by using the operation input unit 20. In accordance with this operation, the CPU 11 assigns an identifier corresponding to the measurement region selected in step S3 and the measurement item selected in step S4 to the measurement result and causes the memory unit 14 to store the resultant data according to the DICOM-SR standard (step S5). That is, in step S5, the CPU 11 functions as an identifier assignment processing unit for assigning an identifier to the measurement result obtained by fundamental measurement based on its clinical meaning according to a predetermined medical communication standard.

Note that in step S5, the CPU 11 may transmit the measurement result assigned with the identifier to a predetermined report server connected to the ultrasonic diagnostic apparatus. When further performing fundamental measurement, the apparatus repeats the processing from step S1 to step S5 (step S6).

As has been described above, according to the first embodiment, it is possible to provide an ultrasonic diagnostic apparatus and medical image processing apparatus which can assign an identifier (assign a clinical meaning) to the data acquired by fundamental measurement by simple processing according to a medical communication standard (e.g., the DICOM-SR standard).

That is, according to the ultrasonic diagnostic apparatus and medical image processing apparatus according to the first embodiment, the user can assign a clinical meaning (assign an identifier) to a fundamental measurement result by only performing very simple processing on the “identifier assignment processing window W”, and can store the measurement result according to the DICOM-SR standard as in the case of application measurement.

More specifically, the user can assign an identifier to a fundamental measurement result according to the DICOM-SR standard by only selecting a measurement region and measurement item associated with the fundamental measurement from the menu items of the pull-down menus of the “measurement region setting portion w1” and “measurement item setting portion w2”.

Furthermore, the apparatus presents the user with measurement items displayed as the pull-down menu of the measurement item setting portion w2 upon narrowing measurement items down to those corresponding to the measurement region selected by the measurement region setting portion w1. This makes it possible to select measurement items very efficiently.

Note that it is possible to change the display order to the menu items of the pull-down menus of the “measurement region setting portion w1” and “measurement item setting portion w2” in accordance with rough region designation by the user at the start of examination or the like. For example, it is possible to display menu items in descending order of estimation probability.

### Second Embodiment

An ultrasonic diagnostic apparatus and medical image processing apparatus according to the second embodiment will be described below. To avoid a redundant description, differences from the ultrasonic diagnostic apparatus and medical image processing apparatus according to the first embodiment will be described.

In the first embodiment described above, the user performs clinical meaning assignment (identifier assignment) processing for a measurement region and measurement item by selecting operation from the menu items of the pull-down menus of the measurement region setting portion w1 and measurement item setting portion w2 in the identifier assignment processing window W. The second embodiment is configured to automate the processing of assigning clinical meanings (assigning identifiers) to measurement regions and measurement items by using a pattern matching technique.

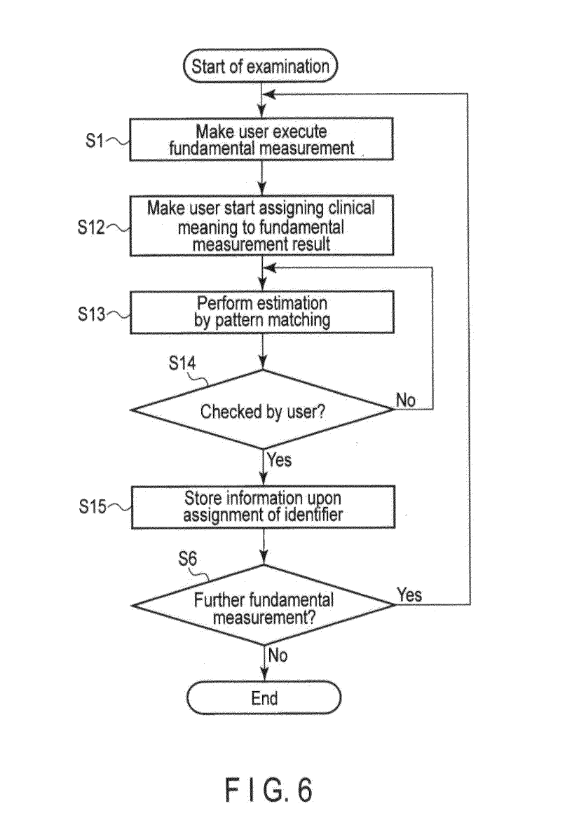

FIG. 6 is a flowchart of the fundamental measurement processing executed by the ultrasonic diagnostic apparatus according to the second embodiment.

First of all, as in the first embodiment, the user executes “fundamental measurement” on an observation region (step S1).

Subsequently, the user performs the operation of starting clinical meaning assignment (identifier assignment) processing for a fundamental measurement result by using an operation input unit 20 (step S12). In accordance with this operation, a CPU 11 (an estimation unit (a measurement region estimation unit and a measurement item estimation unit)) estimates a measurement region on the ultrasonic image by a pattern matching technique (pattern matching processing) (step S13). Estimation processing for a measurement region by the pattern matching technique will be described in detail below. Note that the technique to be used for estimation processing is not limited to the above pattern matching technique, and an arbitrary pattern recognition processing (statistical pattern recognition) may be used. When further performing fundamental measurement, the CPU repeats the processing from step S1 to step S5 (step S6).

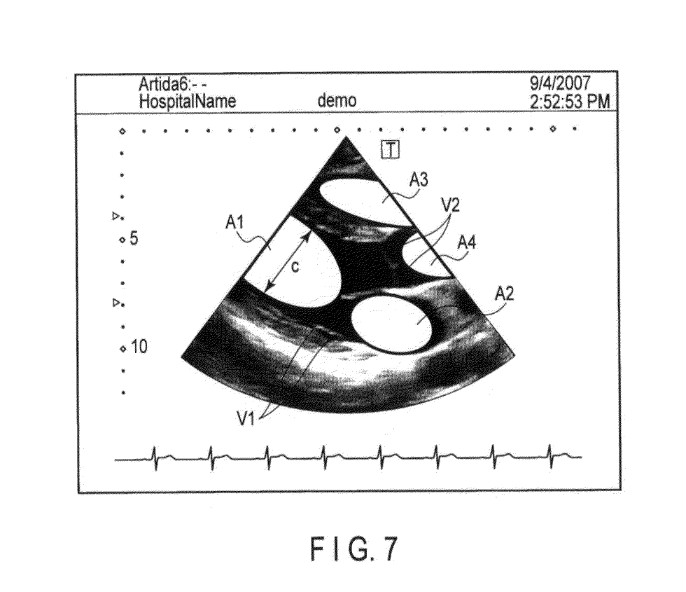

FIG. 7 is a view showing an example of estimation processing for a measurement region by the pattern matching technique. The case shown in FIG. 7 is an example of processing a 4-chamber image (an image indicating a left ventricle posterior wall thickness, left ventricle inner diameter, interventricular septal thickness, and right ventricle inner diameter) at cardiac end-diastole as in the case shown in FIG. 3 described in the first embodiment.

More specifically, the CPU 11 estimates missing black regions based on the “positions” and “sizes (ratios)”. In the case shown in FIG. 7, the CPU 11 estimates regions A1, A2, A3, and A4 as the left ventricle, left atrium, right ventricle, and aorta, respectively. The CPU 11 then estimates a distance c as the “left ventricle inner diameter” because the distance corresponds to the distance inside the region A1 estimated as the left ventricle.

Note that when estimating the regions A1 and A2 as the left ventricle and the left atrium, respectively, the CPU 11 can estimate a region V1 existing between the regions A1 and A2 as the mitral valve. Likewise, when estimating the regions A1 and A4 as the left ventricle and the aorta, respectively, the CPU 11 can estimate a region V2 existing between the regions A1 and A4 as the aortic valve. It is therefore possible to estimate the distance c as the “left ventricle inner diameter” also from the positional relationship between the mitral valve and the aortic valve.

The CPU 11 displays the result obtained by estimating a measurement region and a measurement item by the pattern matching technique as in the case described above on a monitor 40, thereby notifying the user of the result. The user recognizes the estimation result by observing the monitor 40 and decides whether to confirm the estimation result by using the operation input unit 20 (step S14).

In this case, when the user performs the operation of confirming the estimation result (YES in step S14), the CPU 11 assigns the measurement result with an identifier corresponding to the measurement region and measurement item indicated by the estimation result and causes a memory unit 14 to store the resultant data according to the DICOM-SR standard (step S15). Note that in step S15, the CPU 11 may transmit the measurement result assigned with the identifier to a predetermined report server.

When the user performs the operation of not confirming the estimation result (NO in step S14), the process advances to step S13, in which the CPU 11 estimates a measurement region on the ultrasonic image by the pattern matching technique again.

As described above, according to the second embodiment, it is possible to provide an ultrasonic diagnostic apparatus and medical image processing apparatus which can assign an identifier (assign a clinical meaning) to the data acquired by fundamental measurement with very simple processing according to a medical communication standard (e.g., the DICOM-SR standard).

That is, the ultrasonic diagnostic apparatus and medical image processing apparatus according to the second embodiment automatically estimate a measurement region and a measurement item with respect to the data acquired by fundamental measurement and assign an identifier according to the DICOM-SR standard based on the estimation result.

This allows the user to save the trouble of setting a measurement region and a measurement item. In addition, since the user can check the validity of an estimation result, the user can maintain the final estimation accuracy at a desired level even in automatic estimation. That is, the user can assign a clinical meaning to a necessary measurement result while performing fundamental measurement with simple operation, can discriminate the measurement result even by report servers available from other makers in the same way as data acquired by application measurement, and can create a clinical report.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An ultrasonic diagnostic apparatus comprising:
an image acquisition unit configured to acquire an ultrasonic image representing information inside an object;
a measurement unit configured to execute measurement on the ultrasonic image;
an estimation unit configured to estimate a measurement region associated with the measurement based on the ultrasonic image; and
a display unit configured to display the estimated measurement region together with the ultrasonic image.

2. The ultrasonic diagnostic apparatus of claim 1, wherein the estimation unit is configured to estimate a measurement item associated with the measurement based on the ultrasonic image.

3. The ultrasonic diagnostic apparatus of claim 1, wherein the estimation unit estimates the measurement region by pattern matching processing using the ultrasonic image.

4. An ultrasonic diagnostic apparatus comprising:
an image acquisition unit configured to acquire an ultrasonic image representing information inside an object;
a display unit configured to display the ultrasonic image;
a measurement unit configured to execute measurement on the ultrasonic image displayed on the display unit; and
an identifier assignment processing unit configured to assign an identifier to a measurement result obtained by the measurement according to a predetermined medical communication standard based on a clinical meaning of the measurement result.

5. The ultrasonic diagnostic apparatus of claim 4, wherein the identifier assignment processing unit includes:
a measurement region setting unit configured to present a candidate of a measurement region to a user so as to allow the user to set the measurement region with respect to the measurement result;
a measurement item setting unit configured to present a candidate of a measurement item to the user so as to allow the user to set the measurement item with respect to the measurement result; and
a processing unit configured to assign the identifier to the measurement result according to the predetermined medical communication standard based on the measurement region and measurement item set by the user.

6. The ultrasonic diagnostic apparatus of claim 5, further comprising an examination initial setting unit configured to perform initial setting associated with an examination on the object in acquisition of the ultrasonic image,
wherein the measurement region setting unit is configured to present a measurement region corresponding to the initial setting as the candidate to the user in acquisition of the ultrasonic image associated with the measurement.

7. The ultrasonic diagnostic apparatus of claim 5, wherein the measurement item setting unit is configured to present a measurement item corresponding to the measurement region set by the measurement region setting unit as a candidate of the measurement item to the user.

8. The ultrasonic diagnostic apparatus of claim 5, wherein the measurement item setting unit includes:
a measurement region estimation unit configured to estimate the measurement region indicated on the ultrasonic image by pattern matching;
a measurement item estimation unit configured to estimate the measurement item based on an estimation result obtained by the measurement region estimation unit; and
a processing unit configured to assign the identifier to the measurement result of the measurement according to the predetermined medical communication standard using the estimated measurement region and measurement item.

9. The ultrasonic diagnostic apparatus of claim 4, wherein the predetermined medical communication standard includes a DICOM-SR standard.

10. A medical image processing apparatus comprising:
a measurement unit configured to execute measurement on a medical image representing information inside an object;
an estimation unit configured to estimate a measurement region associated with the measurement based on the medical image; and
a display unit configured to display the estimated measurement region together with the medical image.

11. The medical image processing apparatus of claim 10, wherein the estimation unit is configured to estimate a measurement item associated with the measurement based on the medical image.

12. The medical image processing apparatus of claim 10, wherein the estimation unit is configured to estimate the measurement region by pattern matching processing using the medical image.

13. A medical image processing apparatus comprising:
a measurement unit configured to execute measurement on a medical image representing information inside an object; and
an identifier assignment processing unit configured to assign an identifier to a measurement result obtained by the measurement according to a predetermined medical communication standard based on a clinical meaning of the measurement result.

14. The medical image processing apparatus of claim 13, wherein the identifier assignment processing unit includes:
a measurement region setting unit configured to present a candidate of a measurement region to a user so as to allow the user to set the measurement region with respect to the measurement result;
a measurement item setting unit configured to present a candidate of a measurement item to the user so as to allow the user to set the measurement item with respect to the measurement result, and
a processing unit configured to assign the identifier to the measurement result of the measurement according to the predetermined medical communication standard using the measurement region and the measurement item set by the user.

15. The medical image processing apparatus of claim 14, wherein the measurement item setting unit is configured to present the measurement item corresponding to the measurement region set by the measurement region setting unit to the user.

16. The medical image processing apparatus of claim 13, wherein the measurement item setting unit includes:
a measurement region estimation unit configured to estimate the measurement region on the ultrasonic image by pattern matching;
a measurement item estimation unit configured to estimate the measurement item based on an estimation result obtained by the measurement region estimation unit; and
a processing unit configured to assign the identifier to the measurement result of the measurement according to the predetermined medical communication standard using the estimated measurement region and measurement item.
